(19) United States
(12) Patent Application Publication
(10) Pub. No.: US 2015/0182132 A1
(43) Pub. Date: Jul. 2, 2015

(54) MOBILE DEVICE SYSTEM FOR MEASUREMENT OF CARDIOVASCULAR HEALTH

(71) Applicant: CNV Systems Ltd., Vancouver (CA)

(72) Inventors: Paul Ronald Harris, Vancouver (CA); Ji Feng Li, Burnaby (CA)

(21) Appl. No.: 14/420,375

(22) PCT Filed: Feb. 4, 2013
(86) PCT No.: PCT/CA2013/000096

§ 371 Date: Feb. 9, 2015

Related U.S. Application Data
(60) Provisional application No. 61/682,084, filed on Aug. 10, 2012.

(51) Int. Cl. A61B 5/02; A61B 5/00; A61B 5/0402; A61B 5/021; A61B 5/024

(52) U.S. Cl. CPC A61B 5/02007; A61B 5/02125; A61B 5/02416; A61B 5/0402; A61B 5/7275; A61B 5/7282; A61B 5/0022; A61B 5/6823; A61B 5/6898

(57) ABSTRACT

A system that continuously monitors cardiovascular health using an electrocardiography (ECG) source synchronized to an optical (PPG) source, without requiring invasive techniques or ongoing, large-scale external scanning procedures. The system includes an ECG signal source with electrodes contacting the skin, which generates a first set of information, and a mobile device having a camera which acts as a PPG signal source that generates a second set of information. Together with the mobile device's processor, configured to receive and process the first and second sets of information, from which the time differential of the heart beat pulmonary pressure wave can be calculated, continuous data related to cardiovascular health markers such as arterial stiffness can be determined. Variations of the ECG source may include a chest strap, a plug-in adaptor for the mobile device, or electrodes built into the mobile device.

### RELATED APPLICATION

The present application claims the benefit of priority to U.S. Provisional Application No. 61/682,084, filed Aug. 10, 2012.

### PATENT REFERENCES CITED

U.S. Pat. No. 8,239,009
U.S. Pat. No. 8,162,841
U.S. Pat. No. 7,674,231

### US 2004/0030261

U.S. Pat. No. 7,179,228
U.S. Pat. No. 7,479,111
U.S. Pat. No. 7,481,772
U.S. Pat. No. 7,544,168
U.S. Pat. No. 7,803,120
U.S. Pat. No. 7,993,275
U.S. Pat. No. 7,029,447
U.S. Pat. No. 6,736,789
U.S. Pat. No. 6,331,162

### WO 2012/092303

### OTHER REFERENCES

Boutouyrie, Pierre et al. “Obtaining arterial stiffness indices from simple arm cuff measurements: the holy grail?” Journal of Hypertension 2009, 27:2159-2161.
Conlon et al. “Development of a Mathematical Model of the Human Circulatory System” Annals of Biomedical Engineering, Vol. 34, No. 9, September 2006, pp. 1400-1413.
Douniama, C. et al. “Blood Pressure Tracking Capabilities of Pulse Transit Times in Different Arterial Segments: A Clinical Evaluation” Computers in Cardiology, 2009; 36: 201-204.
Fung et al. “Continuous Noninvasive Blood Pressure Measurement by Pulse Transit Time” Proceedings of the 26^{th} Annual International Conference of the IEEE EMBS, San Francisco, Calif., USA, Sep. 1-5, 2004.
Gesche, Heiko et al. “Continuous blood pressure measurement by using the pulse transit time: comparison to a cuff-based method” Eur Appl Physiol, DOI 10.1007/s00421-011-1983-3, Oct. 22, 2010.
Gibbs, Peter and Asada, H. Harry “Reducing Motion Artifact in Wearable Bio-Sensors Using MEMS Accelerometers For Active Noise Cancellation” 2005 American Control Conference, Portland, Oreg., USA, Jun. 8-10, 2005.
Hey, Stefan et al. “Continuous noninvasive Pulse Transit Time Measurement for Psycho-physiological Stress Monitoring” University of Karlsruhe, House of Competence, RG hiper.campus; University of Karlsruhe, Institute for Information Processing Technology; Karlsruhe, Germany.
Kounalakis, S N and Geladas, N D “The role of pulse transit time as an index of arterial stiffness during exercise” Cardiovasc Eng. 2009 September; 9(3):92-7 Epub 2009 Aug. 6.
Liu, Yinbo and Zhang, Y. T. “Pulse Transit Time and Arterial Blood Pressure at Different Vertical Wrist Positions” Manuscript, Joint Research Centre for Biomedical Engineering Department of Electronic Engineering, The Chinese University of Hong Kong, Hong Kong, Jun. 16, 2006.
Naidu, Madireddy U R et al. “Validity and reproducibility of arterial pulse wave velocity measurement using new device with oscillometric technique: A pilot study” BioMedical Engineering OnLine 2005, 4:49 doi:10.1186/1475-925X-4-49.
Oliver, James J. and Webb, David J. “Noninvasive Assessment of Arterial Stiffness and Risk of Atherosclerotic Events” Arteriosclerosis, Thrombosis, and Vascular Biology: Journal of the American Heart Association, 2003; 23: 554-566.
Shaltis, Phillip Andrew “A Wearable Blood Pressure Sensor Using Oscillometric Photoplethysmography and Micro Accelerometers” Submitted to the Department of Mechanical Engineering in Partial Fulfillment of the Requirements for the Degree of Doctor of Philosophy in Mechanical Engineering at the Massachusetts Institute of Technology, June 2007.
Smith, Robin P. et al. “Pulse transit time: an appraisal of potential clinical applications” Thorax, 1999; 54: 452-457.
Zhang, Qiao “Cuff-Free Blood Pressure Estimation Using Signal Processing Techniques” Thesis: College of Graduate Studies and Research; Biomedical Engineering; University of Saskatchewan; August 2010.

### FIELD OF THE INVENTION

The present invention is in the field of diagnosis and monitoring of cardiovascular health.

### BACKGROUND OF THE INVENTION

Cardiovascular disease is the leading killer in the world and represents a major global health problem. It encompasses a wide gamut of disorders involving the heart and blood vessels that are typically linked. In the developed world, cardiovascular conditions such as stroke and myocardial infarction are leading killers directly caused by atherosclerosis, arterial stiffness, and hypertension.

As with any disease, prevention is far and away the better choice than post-crisis treatment, whenever possible. Annually, billions of dollars around the world could be saved in healthcare costs if cardiovascular conditions were managed before becoming life-threatening. Standards of living, particularly for the elderly, would also dramatically improve if cardiovascular conditions were caught and prevented at an early stage. To prevent cardiovascular disease, traditional diagnosis and monitoring methods have revolved around blood pressure (BP).

Conventional non-invasive blood pressure monitoring involves cuff sphygmomanometry, wherein an inflatable cuff is used to restrict blood flow, such as in the arm, and a measuring unit is used to determine at what pressure blood flow is just starting and at what pressure it is unimpeded. While relatively simple to use, cuff sphygmomanometry can be uncomfortable, and only generates one point of data at the time of use. It can also be inaccurate for various reasons, including emotional state at the time of use, time of day, and user judgment error.

Invasive means for blood pressure monitoring typically involve an intra-arterial catheter. While more accurate and capable of continuously monitoring blood pressure, invasive means run the serious risks of arterial injury and infection.

Photoplethysmography (PPG) is a modern technique for optically detecting blood volume changes in blood vessels. Typically, with transmission PPG, a photodiode emits infrared light through a small part of the body such as a finger or thumb, and that emission is detected on the other side of that small body part. Changes in light absorption are detected and can be used to measure blood flow, blood content, and other circulatory conditions.

As opposed to transmissive light that illuminates through an area of body tissue, a PPG sensor may instead be reflective. Reflectance PPG has significantly greater technical challenges to overcome, but the notable advantage of reflectance PPG over transmission PPG is the ability to place the emitter and the adjacent sensor to detect blood volume changes almost anywhere on the body that has blood vessels.

Many modern devices and techniques for monitoring cardiovascular health in the prior art focus on fixed BP values. However, BP in and of itself does not provide a complete measure for a particular individual's health, as BP values and health effects vary greatly between individuals. Also, it is difficult to achieve truly precise fixed systolic/diastolic values using the BP measuring devices known in the art. These devices tend to provide estimates only, and are non-continuous, momentary spot measurements that are subject to the effects of the individual's emotional and environmental circumstances at the time the measurement is taken.

Algorithms for estimating blood pressure using pulse transit time are known in the art, such as Fung et al. “Continuous Noninvasive Blood Pressure Measurement by Pulse Transit Time” Proceedings of the 26^{th} Annual International Conference of the IEEE EMBS, San Francisco, Calif., USA, Sep. 1-5, 2004.

Many doctors would agree that a more accurate indicator than a fixed BP value for any particular individual's cardiovascular health is a measure for arterial stiffness, or the elasticity of an individual's arterial walls.

Arterial stiffness has been previously measured using both invasive and non-invasive methods. Non-invasive means tend to fit into three types: 1) measuring Pulse Wave Velocity (PWV), such as with Doppler ultrasound, applanation tonometry, or MRI, 2) relating change in diameter (or area) of an artery to distending pressure, such as with ultrasound or MRI, or 3) assessing arterial pressure waveforms, such as with applanation tonometry. Such methods, and comparisons between them, can be found in Oliver, James J. and Webb, David J. “Noninvasive Assessment of Arterial Stiffness and Risk of Atherosclerotic Events” Arteriosclerosis, Thrombosis, and Vascular Biology: Journal of the American Heart Association, 2003; 23: 554-566.

Both pulse transit time (PTT) and pulse wave velocity (PWV) have been suggested for assessment of arterial stiffness, such as in Boutouyrie, Pierre et al. “Obtaining arterial stiffness indices from simple arm cuff measurements: the holy grail?” Journal of Hypertension 2009, 27:2159-2161 and Kounalakis, S N and Geladas, N D “The role of pulse transit time as an index of arterial stiffness during exercise” Cardiovasc Eng. 2009 September; 9(3):92-7 Epub 2009 Aug. 6.

Health monitoring via smartphones and other mobile devices is a burgeoning industry, with commercially available apps and hardware such as those by iHealth Lab, Inc. and Azumio, Inc. AliveCor has also produced a smartphone ECG which incorporates electrodes into a wireless case that snaps onto the back of a smartphone.

Bluetooth heart rate detecting chest straps that communicate with smartphones are known in the art as well, and are commercially available. Examples include those available by Kyto Electronic Co., Ltd. and Dayton Industrial Co., Ltd.

Numerous devices or systems that utilize ECG and PPG to measure BP, PTT or other cardiovascular indicators are also known in the art, such as in the following patents:

U.S. Pat. No. 7,029,447 claims a specific method and system of measuring BP using an ECG and peripheral PPG to derive pulse wave transit time.

U.S. Pat. No. 7,479,111 claims a method for measuring arterial BP through pulse transit time, using an ECG signal and a PPG signal, and compensating for other factors such as sensor contact force, nervous activities, cardiac output, and ambient temperature.

U.S. Pat. No. 7,481,772 claims a system for continuously monitoring BP through pulse transit time calculations. This system comprising a patch sensor that attaches to a patient's skin, which has an optical sensor and an electrode sensor surrounding the optical sensor, and is in communication with a separate processing component.

U.S. Pat. No. 7,674,231 claims a method of deriving an output circulatory metric, such as BP, by calculating a pulse transit time (PTT) between a first and second plethysmographic signals.

U.S. Pat. No. 7,803,120 and U.S. Pat. No. 7,993,275 claim a method and a device, respectively, for measuring BP comprising two optical sensors, two electrodes, and a microprocessor to derive three pulse transit times to determine BP.

US 2004/0030261 discloses a method and system for non-invasively measuring BP using an ECG and a peripheral PPG sensor to measure pulse wave velocity, and through that measurement estimating BP. Calibration via cuffed BP measurements is also disclosed.

U.S. Pat. No. 7,179,228 claims a device and method for measuring BP comprising a first optical module that generates a first set of information, a second optical module that generates a second set of information, an electrical sensor with an electrode pair that generates a third set of information, and a processor that calculates BP using the three sets of information.

U.S. Pat. No. 7,544,168 claims a cuff-based BP-measuring device used in conjunction with a PPG sensor. The inflatable cuff is needed for the BP measurement itself, or at the very least for calibration purposes.

U.S. Pat. No. 8,162,841 discloses and claims a non-implantable surface ECG and surface PPG system for measuring blood pressure. However, their invention entirely centres around their subcutaneously implanted device, with a simple statement that the implanted technology can be used on the surface as well. Those skilled in the art of photoplethysmography know that surface PPG has a significant range of issues that must be overcome for a viable diagnostic and monitoring device to be made. Noise, accuracy, light shielding, calibration, and wearer movement are just a sampling of the issues of surface PPG that are not discussed or enabled in this patent.

U.S. Pat. No. 6,736,789 discloses and claims a blood treatment device which may comprise an ECG device and a PPG device to measure pulse transit time or pulse wave velocity.

U.S. Pat. No. 6,331,162 discloses and claims a method of measuring pulse wave velocity using two PPG probes on a patient's back, coupled with an ECG.

WO 2012/092303 discloses and claims a system for measuring stroke volume and cardiac output comprising an impedance sensor with electrodes, an optical sensor with an optical probe and optical circuit, and a processing system. The impedance sensor with electrodes is a chest-worn ECG, the optical sensor with an optical probe and optical circuit is a transmission PPG device worn on the thumb, and the processing system is a wrist-worn transceiver.

U.S. Pat. No. 8,239,009 discloses and claims a biosignal measurement module comprising detection electrodes, a photo detector, a pose detector, and a processor, for calculating height-dependent pulse transit time, from which blood pressure can be determined. The module may be integrated into a mobile phone, although no mention is made of how or to what purpose.

It remains highly desirable to have a means for continuous cardiovascular health monitoring, particularly for arterial stiffness, that is simple, affordable, portable, convenient, accurate, non-invasive, and compatible with modern computer and communications technology. Also, examples of current PPG devices still show the need for a simple, synchronous, portable, convenient, data-transferrable PPG sensor system.

### SUMMARY OF THE INVENTION

The present invention provides a system that continuously monitors cardiovascular health using an electrocardiography (ECG) source synchronized with a mobile device camera photoplethysmography (PPG) source, without requiring invasive techniques or ongoing expensive, large-scale external scanning procedures. The system includes an ECG signal source that generates a first set of information. A mobile device having a camera that functions as a PPG signal source generates a second set of information. A processor, housed within the mobile device, is configured to receive and process the first and second sets of information, from which the pulse transit time (PTT) of the heart beat pulmonary pressure wave can be calculated. Continuously monitored PTT can be used as a marker of cardiovascular health itself, or it can be used to calculate or estimate other cardiovascular health markers such as pulse wave velocity (PWV), blood pressure (BP), or arterial stiffness.

The components of the system can collect synchronized measurements on continuous basis over an extended period of time so as to determine trending of an individual's personal cardio vascular markers over time, diminishing the requirement for secondary calibration with outside systems. The system is designed with portability and ease of use during regular, daily-life activities in mind. There are no external wires, and the system is centred around a common mobile device having a camera and a processor, such as a smartphone. Such monitoring over time also allows for sustained biometric measurements, leading to clarification of an individual user's biometric signature, from which abnormalities in the rate of circulatory degeneration can be determined and the application of preventive measures before a health crisis occurs.

The mobile device may also have an accelerometer to monitor motion and signal noise. When such noise is detected to such a level as to render the PPG data defective, the synchronized system can be programmed to accept and display only accurate data from the ECG source until the PPG source is determined to be accurate again.

The invention also provides a method of use for the mobile device system to monitor, either continuously or intermittently, one or more of arterial stiffness, blood pressure, heart rate, pulse transit time, and pulse wave velocity.

### BRIEF DESCRIPTION OF THE DRAWINGS

The following figures for embodiments of the invention are meant to be read in conjunction with the detailed description:

FIG. 1 depicts a front view of a user wearing a chest strap ECG source and holding a smartphone, having a camera which functions as a PPG source, in a hand.

FIG. 2 depicts a front view of a chest strap ECG source.

FIG. 3 depicts a front view of a smartphone having a camera, which functions as a PPG source.

FIG. 4 depicts a front view of a hand holding a smartphone with a finger over the camera CCD and LED flash, while the smartphone is in Bluetooth communication with a chest strap ECG source.

FIG. 5 depicts a front view of an ECG adaptor before and after plugging into the main bus port of a smartphone.

FIG. 6 depicts a front perspective of how an ECG adaptor plugs into a smartphone's main bus port.

FIG. 7 depicts a front view of a user grasping a smartphone with an ECG adaptor plugged into the main bus port, in order to take a pulse transit time reading.

FIG. 8 depicts an internal schematic of an ECG adaptor.

FIG. 9 is a graph depicting a target heart beat and a target pulse wave, as detected by an ECG source and a PPG source, respectively.

FIG. 10 depicts a method of use in block diagram format.

### DETAILED DESCRIPTION OF THE PREFERRED EMBODIMENTS

### Acronyms and Definitions

For the present invention, acronyms and definitions common in the fields of cardiovascular health and mechanical engineering are to apply. Specific terms include the following:

Photoplethysmography (PPG) is the volumetric measurement of tissue using an optical device. The two common forms of PPG are transmission PPG, wherein tissue is irradiated by light and the exiting light intensity is measured by a photodetector on the other side of the tissue from the light emitter, and reflectance PPG, wherein the light emitter and photodetector are placed on the same side of the tissue and reflected light intensity is measured instead.

Electrocardiography (ECG) is the measurement of the electrical activity of the heart, as detected by electrodes. An electrocardiogram (also ECG) is a recording of the electrical activity of the heart, as detected by electrodes attached to or contacting the skin.

Pulse Wave Velocity (PWV) is the velocity at which a pulse wave travels through the arterial tree.

Pulse Transit Time (PTT or T) is the time it takes for a pulse wave to travel between two sites in the arterial tree.

Blood Pressure (BP or P) is the pressure exerted by circulating blood upon the walls of blood vessels. During each heartbeat, blood pressure ranges between a maximum systolic pressure when the heart contracts, and a minimum diastolic pressure when the heart is at rest.

Arterial Stiffness is the stiffness of the arterial walls.

Light Emitting Diode (LED) is a semiconductor light source.

BTLE or BLE stands for Bluetooth low energy, and is a feature of Bluetooth 4.0 wireless radio technology.

LCD stands for liquid crystal display, and is a video display that uses the light modulating properties of liquid crystals.

USB stands for Universal Serial Bus, and is an industry standard for cables, connectors, and communications protocols between computers and electronic devices.

ADC stands for Analog-to-Digital Converter, and is a device that uses sampling to convert a continuous quantity to a discrete time representation in digital form (i.e. it converts an analog signal into a digital signal).

CCD stands for charge coupled device, and is an image sensor type.

CMOS stands for complementary metal-oxide-semiconductor, and is another image sensor type.

App stands for application software, and is computer software designed to help a user perform a specific task or tasks.

### Description

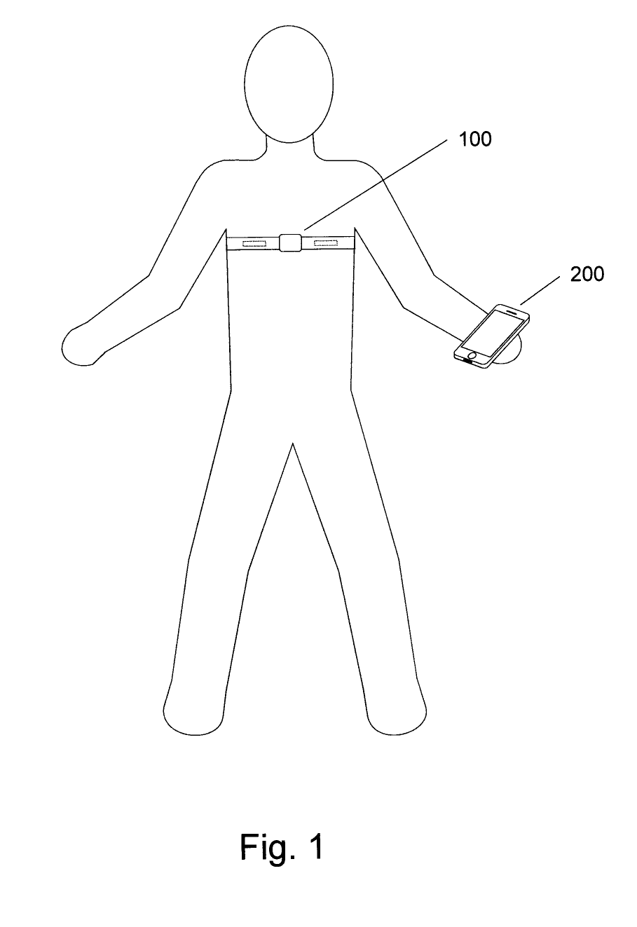

In a first embodiment of the invention, there is a chest strap ECG source (100) strapped around the chest of a person, and a smartphone (200), held in a hand, as shown in FIG. 1.

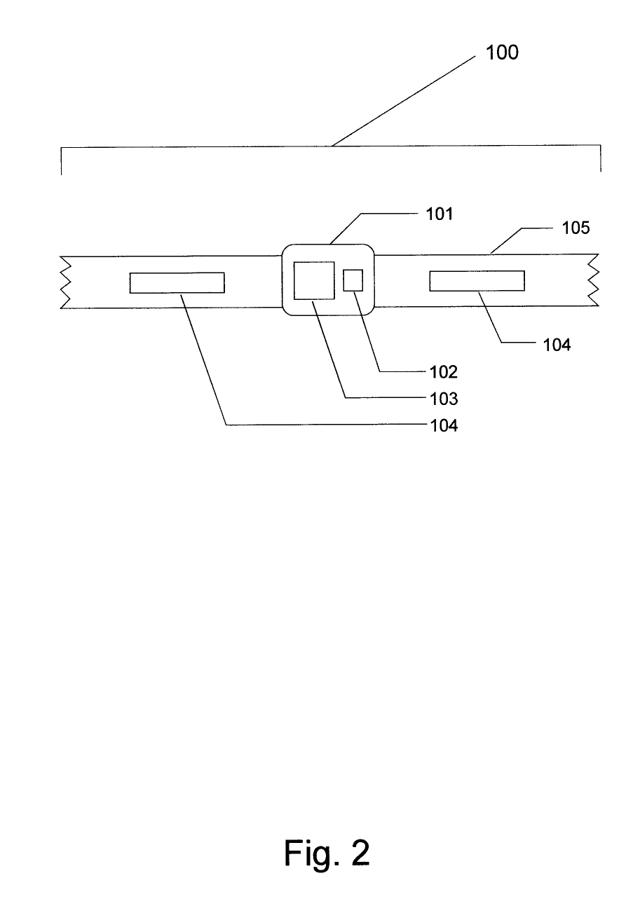

In this first embodiment, the chest strap ECG source (100) has the following components: ECG housing (101), ECG circuitry (103), elastic strap (105), wireless transmitter (102), and electrode contact strips (104), as shown in FIG. 2. The ECG circuitry (103) and wireless transmitter (102) are housed within the centrally located ECG housing (101). The elastic strap (105) extends from two opposing sides of the ECG housing (101) to strap around the wearer. When the ECG source (100) is worn, the electrode contact strips (104) on the elastic strap (105) make contact with the skin of the chest of the wearer.

Such basic ECG chest straps, and similar ones, are known in the art and are commercially available. In the present embodiment, the ECG chest strap is modified with BTLE wireless communication.

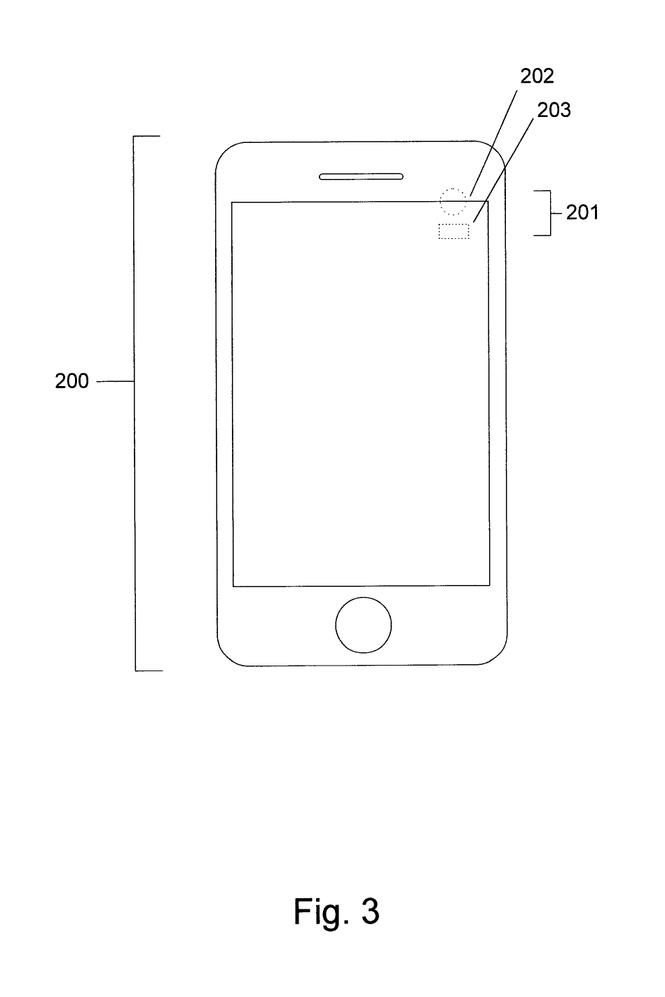

Also in this first embodiment, the smartphone (200) has at least the following components: a camera (201) comprising an image sensor (202) and LED flash (203), as shown in FIG. 3. The smartphone (200) also has a processor that is programmed with an app that converts the smartphone (200) and its camera (201) into a PPG source.

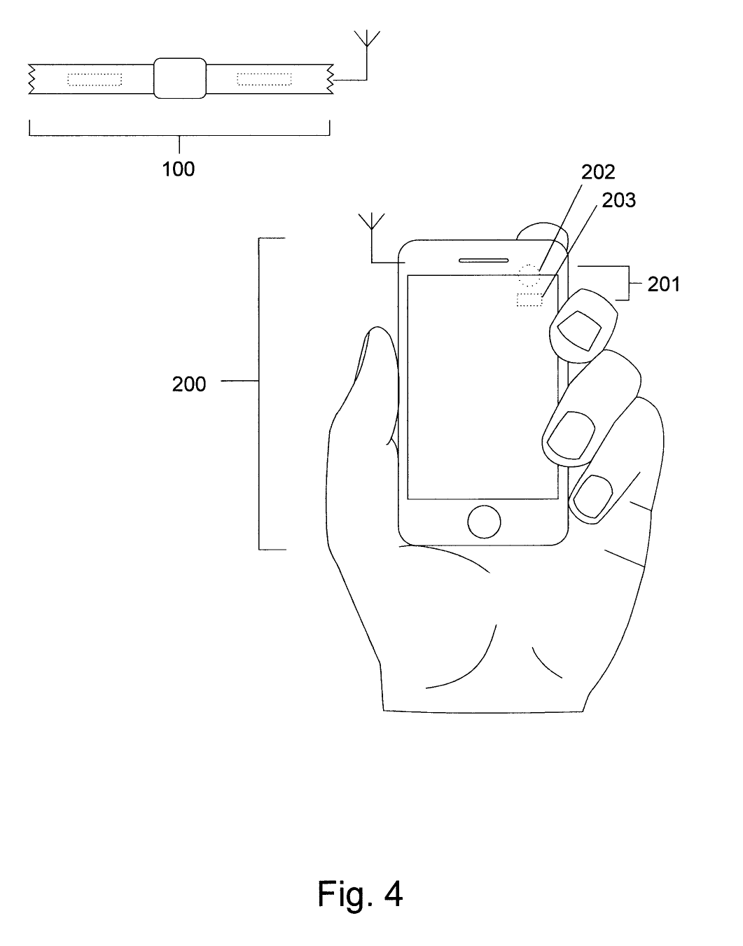

To obtain a pulse transit time reading using the first embodiment, a person first straps the chest strap ECG source (100) around the chest to obtain an ECG signal and data for a target heart beat. The person then holds the smartphone (200), programmed with an app that converts the camera (201) into a PPG source, in a hand. The smartphone (200) and camera (201) are held with a finger over the image sensor (202) and LED flash (203), as shown in FIG. 4, to obtain a PPG signal and data for the pulse wave in the tissue of the finger caused by the same target heart beat. The ECG and PPG data are communicated to the smartphone processor. These two sets of data are synchronized by the processor and used to calculate pulse transit time and related cardiovascular markers.

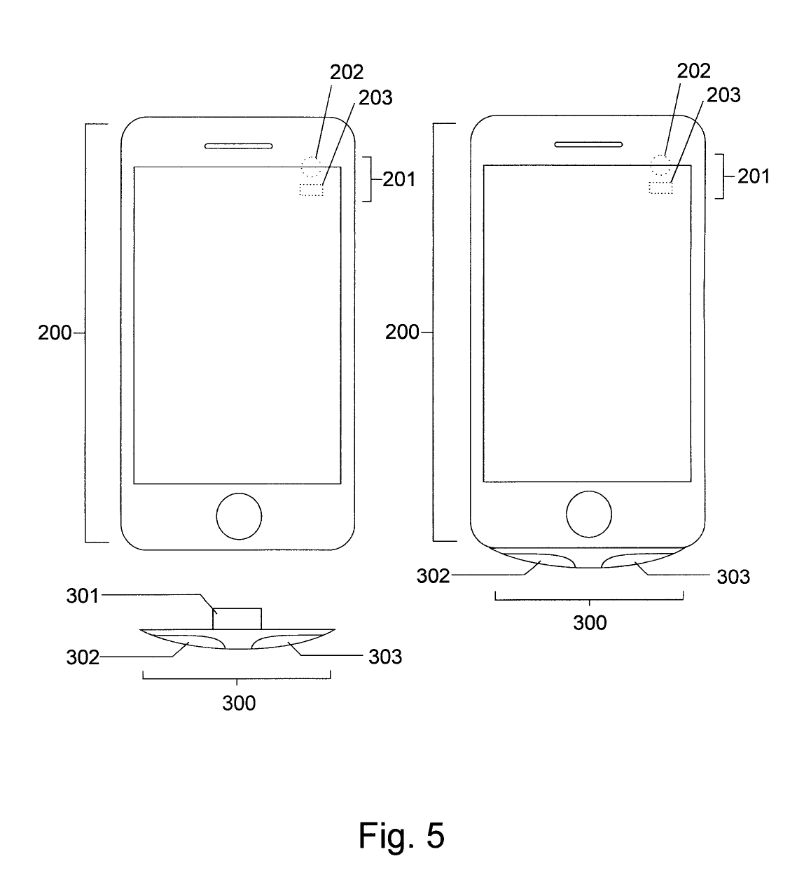

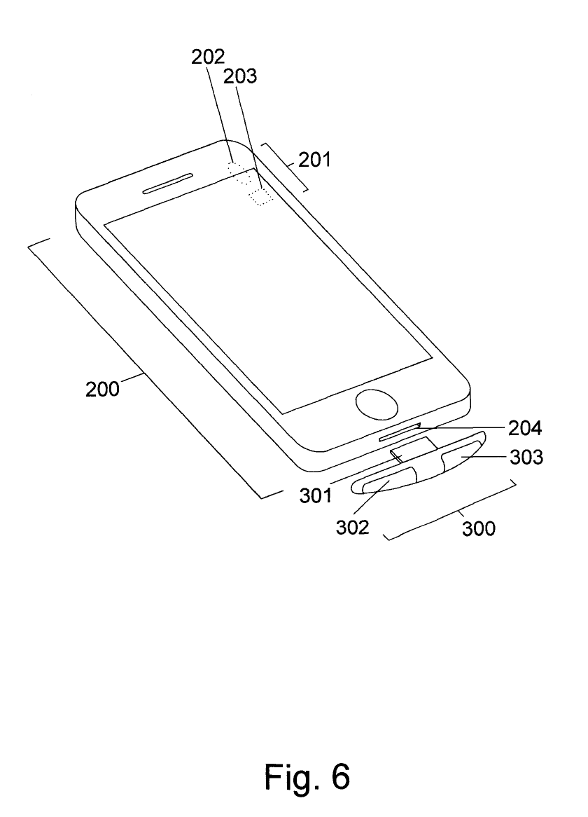

In a second embodiment of the invention, there is a smartphone (200) having a camera (201) and at least one bus port (204). An ECG adaptor (300) is plugged into the bus port (204) of the smartphone (200) via the adaptor's bus connector (301), as shown in FIGS. 5 and 6. On the surface of the adaptor (300) are two separate ECG contact electrodes, a first contact electrode (302) and a second contact electrode (303).

The adaptor (300) may have a plurality of contact electrodes in any position or configuration that allows for the completion of an ECG circuit through the user's body.

In this second embodiment, the smartphone (200) is programmed with an app that recognizes the plugged-in ECG adaptor (300) and also converts the smartphone camera (201) into a PPG source. As with the first embodiment, the smartphone camera (201) has at least the following components: an image sensor (202) and an LED flash (203), as shown in FIGS. 5, 6 and 7.

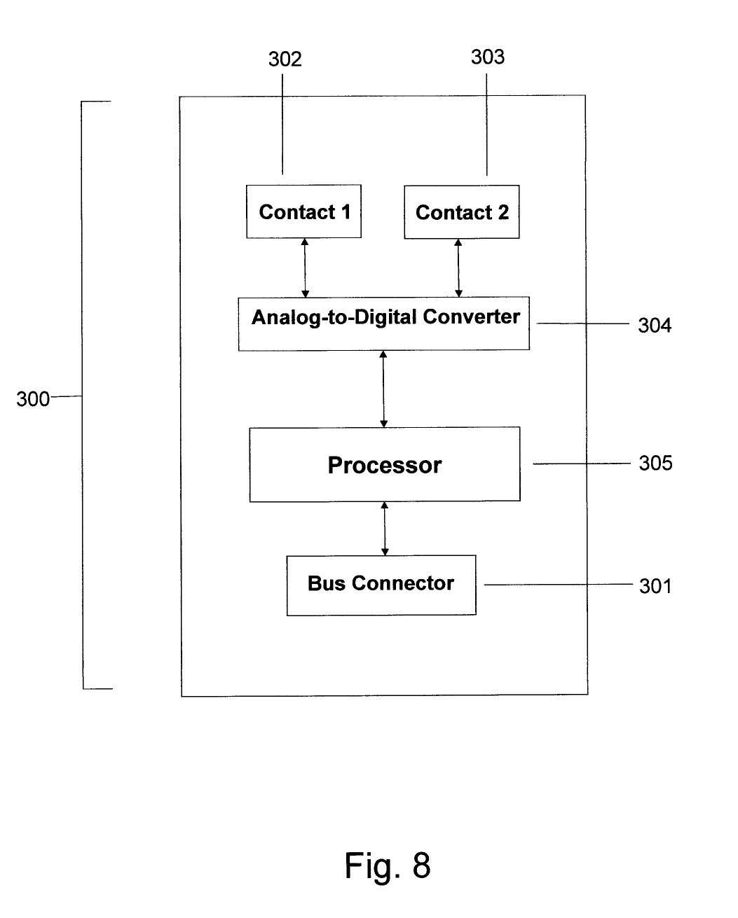

An internal schematic of the ECG adaptor (300) of the second embodiment is shown in FIG. 8. Within the ECG adaptor (300) is a processor (305) that communicates with the adaptor's components, including the bus connector (301) and the analog-to-digital converter (304). The analog-to-digital converter (304) accepts the analog signals from the first contact electrode (302) and the second contact electrode (303) when the adaptor (300) is in use, and converts this ECG data into a digital form for the processor (305). When the bus connector (301) is inserted into the smartphone's bus port (204), the adaptor's processor (305) communicates this digital ECG data to the main processor of the smartphone (200).

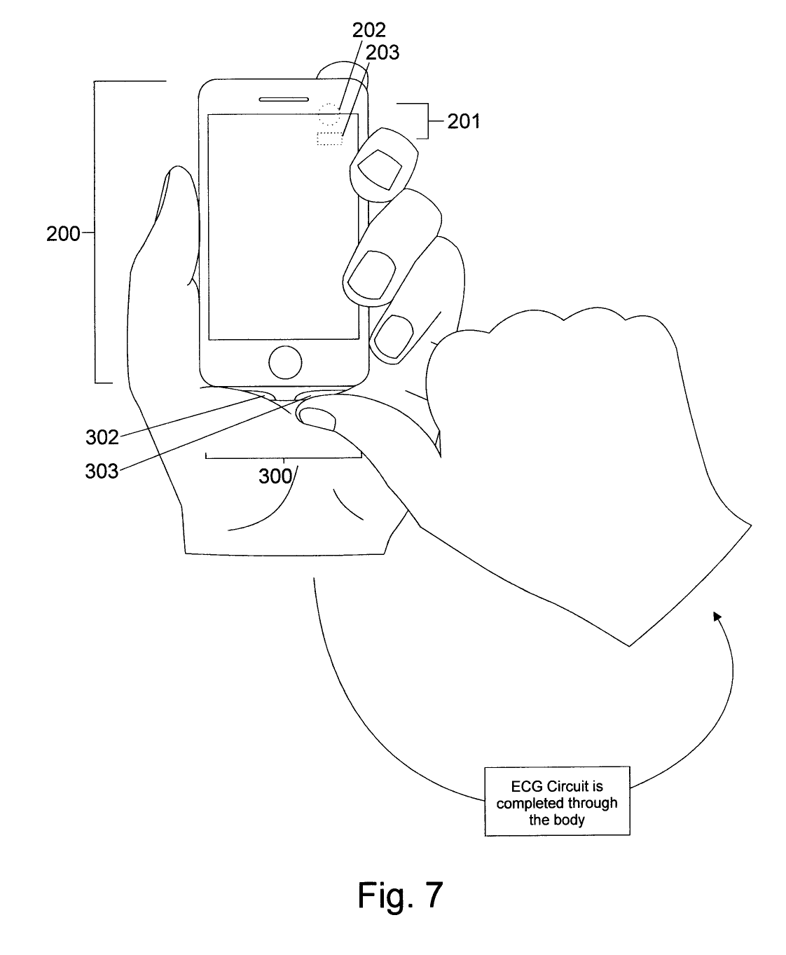

To obtain a pulse transit time reading using the second embodiment, a person first obtains an ECG signal and data for a target heart beat. The person plugs the ECG adaptor (300) into a smartphone (200) programmed with an app that recognizes the adaptor (300), and then the person grasps the combined smartphone (200) and plugged-in adaptor (300) in a manner that contacts the palm of one hand with a first contact electrode (302), and contacts the thumb of the other hand with a second contact electrode (303), as shown in FIG. 7. This completes an ECG circuit through the person's body. The person then holds a finger over the smartphone camera (201), wherein the smartphone (200) has also been programmed with an app that converts the camera (201) into a PPG source. The smartphone camera (201) PPG source is contacted with a finger over both the CCD image sensor (202) and LED flash (203) to obtain a PPG signal and data for the pulse wave in the tissue of the finger caused by the same target heart beat. The ECG and PPG data are communicated to the smartphone main processor. These two sets of data are synchronized by the main processor and used to calculate pulse transit time, from which related cardiovascular markers can be derived.

In a third embodiment, the ECG adaptor is not a separate, detachable component from the mobile device at all. A mobile device may have integrated ECG contact electrodes, a camera that can be converted into a PPG source, and an app that controls and coordinates these ECG and PPG components, for a fully functional pulse transit time (or other cardiovascular marker) monitoring embodiment of the present invention, all contained in a single mobile device.

The PPG source in the present embodiments may be part of any mobile device that has a processor, particularly one programmable with apps, and that has a camera. Such mobiles devices include, but are not limited to, smartphones, cell phones, tablet computers, personal digital assistants (PDA), handheld consoles, digital cameras, and digital camcorders. A handheld console may be a known handheld gaming console, or may be a specially constructed handheld device for the purposes of the present invention.

The image sensor for the mobile device camera may be CCD, CMOS, or any other image sensor type found in modern mobile device cameras.

The mobile device camera LED flash of the present embodiments may emit light at any wavelength known in the art that is suitable for PPG.

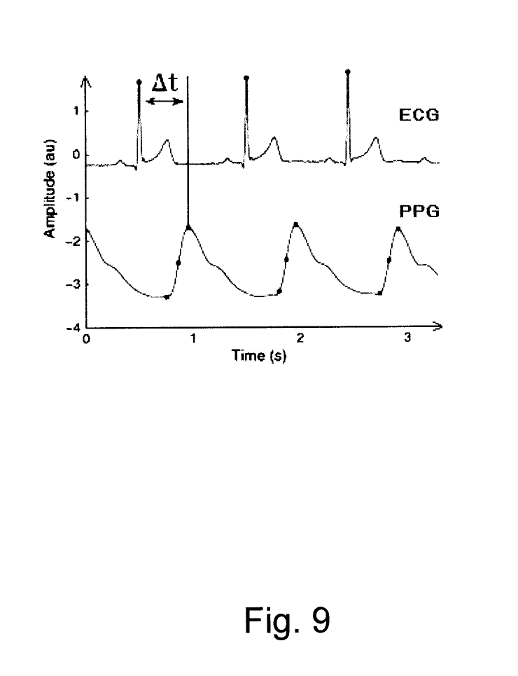

A target heart beat cycle, and its corresponding pulse wave, is shown in FIG. 9. The peak amplitude of a target heart beat is detected by the ECG. The peak amplitude of the corresponding pulse wave in the blood vessels is detected by the PPG. The difference in time between these peak amplitudes is the At or PTT, through which other cardiovascular health markers can be calculated. For example, PWV is the distance traveled by the pulse (which is closely approximated by the distance between the chest strap ECG and mobile device camera PPG sources, or the measured length of the artery from the heart to the finger) divided by PTT (PWV=distance/PTT). Arterial stiffness can be derived from PWV through the Moens-Korteweg equation.

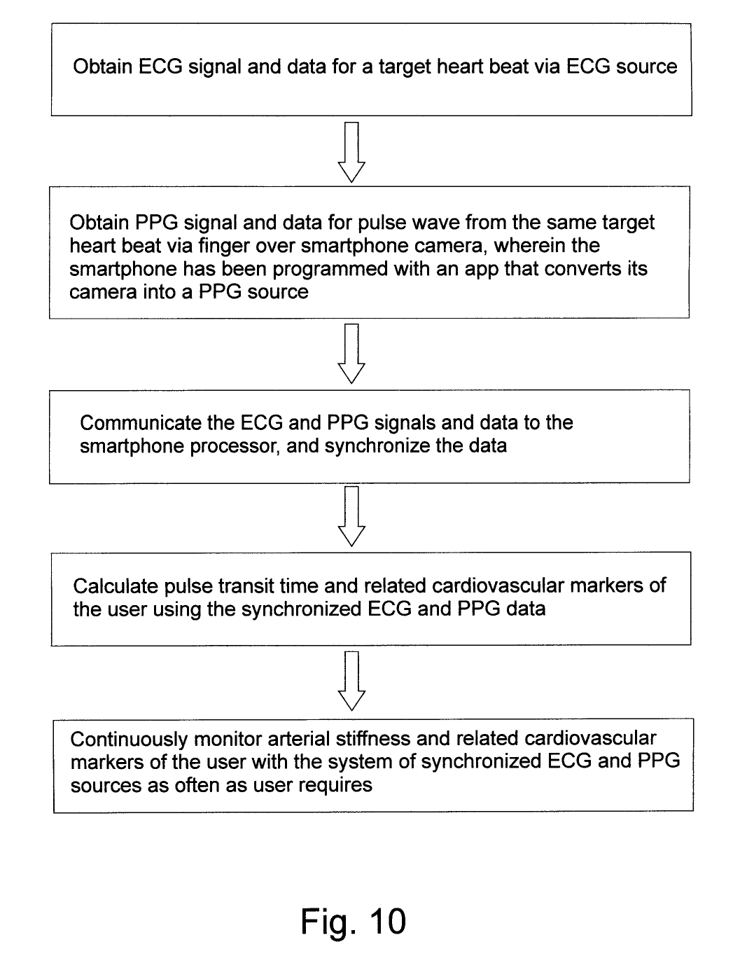

Alternatively, as seen in Zhang, Qiao “Cuff-Free Blood Pressure Estimation Using Signal Processing Techniques” Thesis: College of Graduate Studies and Research; Biomedical Engineering; University of Saskatchewan; August 2010 and Hey, Stefan et al. “Continuous noninvasive Pulse Transit Time Measurement for Psycho-physiological Stress Monitoring” University of Karlsruhe, House of Competence, RG hiper.campus; University of Karlsruhe, Institute for Information Processing Technology; Karlsruhe, Germany, other specific endpoints in the pulse waveform as detected by the PPG (three different endpoints visible in FIG. 10) may be used to determine the At or PTT. Possibilities include, but are not limited to, the peak, the midpoint, the foot, the point of maximal slope, and the virtual basepoint (which corresponds to the intersection point between the tangent to the pulse wave at the point of maximal slope and the horizontal line going through the point having the absolute minimum signal). Different endpoints are suggested to have different advantages in measuring and using the PTT value. For example, using the virtual basepoint has been suggested to give a better virtual noise and artifact robustness. Using the point of maximal slope has been suggested to be strongly related to systolic BP.

In a fourth embodiment, both a chest strap ECG and contact ECG electrodes (whether on a separate plug-in adaptor or integrated into the mobile device itself) are present and used in conjunction with the mobile device camera PPG source to monitor cardiovascular health. The dual ECG sources and single PPG source may be used to calibrate or correct the signal or data from each other, to more accurately determine a cardiovascular health marker such as heart rate, arterial stiffness, blood pressure, pulse transit time, or pulse wave velocity. If one signal is jeopardized, another signal may compensate. Most preferably in this fourth embodiment, the heart rate signal and data for a user is continuously calibrated using this synchronized system of dual ECG and single PPG sources.

Corresponding to the systems of the present embodiments, a general method of use is shown in FIG. 10 in block diagram format. To obtain a pulse transit time reading using these embodiments, a person first obtains an ECG signal and data for a target heart beat. The person then grasps a mobile device, with a finger over the camera PPG source, to obtain a PPG signal and data for the pulse wave in the tissue of the finger caused by the same target heart beat. The ECG and PPG signals and data are communicated to the mobile device processor. These two sets of data are synchronized by the processor and used to calculate pulse transit time and related cardiovascular indicators, such as arterial stiffness. In this manner, it becomes possible to continuously derive measurements relating to arterial stiffness and associated cardiovascular markers with this synchronized set of ECG and PPG sources, unobtrusively and without requirement for any external connecting wires. Output measurements of data stream sequences collected from subsequent date ranges can then be compared to verify cardiovascular trending markers corresponding to a specific individual's rate of arterial stiffness and circulatory degeneration, effectively providing a personalized biometric trending signature, from which preventive measures can be potentially applied before a health crisis occurs.

Optionally, an accelerometer is included with the PPG source in the mobile device to help minimize motion and signal noise. A processor with the accelerometer can be used to detect when excessive noise has occurred so as to render the PPG signal and data defective. This processor then discounts the PPG data, and only the ECG data is relied upon to give cardiovascular marker information such as heart rate, until such time as the accelerometer detects that the noise has diminished and the processor determines that the PPG data is reliable again.

Accelerometry to minimize motion and signal noise is known in the art, such as in Gibbs, Peter and Asada, H. Harry “Reducing Motion Artifact in Wearable Bio-Sensors Using MEMS Accelerometers For Active Noise Cancellation” 2005 American Control Conference, Portland, Oreg., USA, Jun. 8-10, 2005.

In the embodiment of the method shown in FIG. 10, arterial stiffness of the wearer is monitored, but the systems of the present invention can be configured to measure, calculate, or estimate one or more cardiovascular health markers over time, including, but not limited to, arterial stiffness, blood pressure, heart rate, pulse transit time, and pulse wave velocity. In another embodiment, at least two of arterial stiffness, BP, HR, PTT, and PWV are monitored over time. In another embodiment, at least three of arterial stiffness, BP, HR, PTT, and PWV are monitored over time. In another embodiment, all of arterial stiffness, BP, HR, PTT, and PWV are monitored over time. The mobile device processor can be configured to output any or all of these values at the push of a button or the touch of a display screen, simultaneously or separately. Most preferably, a smartphone processor can be programmed to output and display one or more of these cardiovascular health values on the smartphone's LCD display screen.

Wireless transmitters and transceivers suitable for the present invention are known in the art, and preferably utilize Bluetooth technology, although other technologies are possible. The mobile device is preferably USB compatible for computer data transfer and battery recharge purposes, although not limited to such, and other bus port types and other means for data transfer or battery recharge are possible.

Combined signal measurement and data interpolation derived from protracted sequences of continuous monitoring output via the present embodiments negates or offsets the need for secondary calibration with an outside source, such as from a cuff, and it allows for determination of arterial health trending markers over time. Data derived from a continuous measurement process provides for more complete analysis of cardiovascular health indicators beyond intermittent measurements such as BP, enabling derivation of individual biometric trending that can account for anomalous BP, PTT, or PWV values due to moments of stress and other health and environmental triggers.

Continuous, unobtrusive, and portable monitoring in the present embodiments also has strong application for telemedicine purposes. Without limitation, these monitoring embodiment methods could be used to remotely validate rehabilitation compliance or fitness goals. It could be used in the doctor's office, in the hospital, in the home, and as the individual carries out daily activities. It has potential for use not only in the medical and fitness fields, but also for monitoring purposes in health insurance, policing, athletics, and military defense. It could be used to remotely store or selectively display cardiovascular health data about one or more individuals over a period of time, including during healthy or illness stages, and in determining health marker changes due to disease or aging. Those of ordinary skill in the art could identify a range of practical uses for the present embodiments.

With the sustained synchronous and continuous nature of the present embodiments, there is potential for more accurate PPG measurement of arterial performance efficiency, as opposed to the arterial measurement of the prior art.

The systems of the present embodiments offer greater usage convenience and portability than is seen with cardiovascular health monitoring devices known in the art. The present systems are designed with unobtrusive, portable, and continuous daily use in mind, whether it be in combination with a hidden chest strap ECG and mobile device camera PPG, or simply with the mobile device encompassing both contact electrodes and a camera PPG source. The present systems do not interfere with everyday activities, as wired systems, larger systems, heavier systems, or systems requiring less common electronic devices do. The present systems are easily worn or used, in a secure, portable, and unobtrusive manner. The mobile devices involved are extremely common in this day and age.

While reflectance PPG, based on reflected light from the LED flash, is preferred for the mobile device camera, it is possible for the mobile device camera to be a transmission PPG source as well, or a combination of reflective and transmissive from both the LED flash and other ambient light source. So long as sufficient light passes through the tissue of the finger and is detected by the image sensor to render a PPG reading, transmission PPG, or a combination of reflectance and transmission PPG, is possible in the present invention.

A major advantage of the present invention is the ability to continuously monitor cardiovascular health while involving, at most, two discreet, convenient, portable, unobtrusive devices (chest strap ECG and mobile device camera PPG). An even greater advantage arises with the present invention when the mobile device comprises both contact ECG electrodes and the camera PPG source, such that all of the components needed to continuously monitor cardiovascular health are conveniently and unobtrusively housed in a single, compact, handheld device, at a single point on the body.

Links have been suggested throughout the prior art between the values of PTT, PWV, changes in BP, and arterial stiffness. It is generally accepted that both PTT and PWV can be regarded as indices of arterial stiffness, and that both can also be employed as estimators of BP. HR is easily monitored with either the ECG or PPG sources by measuring beats per unit time (typically beats per minute, or bpm). The present invention may be configured to calculate or estimate any or all of arterial stiffness, BP, HR, PTT, and PWV through continuous monitoring.

Much of the prior art is focussed on trying to obtain a fixed BP value for an individual to determine their cardiovascular health. More preferable than trying to calculate only a fixed BP value, though, is to determine the degeneration of arterial elasticity over time. In one embodiment, once pulse transit time is calculated, and pulse wave velocity is derived, a suitable formula for linking pulse wave velocity and arterial stiffness is the Moens-Korteweg equation:

PWV=√[(E_{inc}·h)/(2rρ)].

The Moens-Korteweg equation states that PWV is proportional to the square root of the incremental elastic modulus, (E_{inc}), of the vessel wall given constant ratio of wall thickness, h, to vessel radius, r, and blood density, ρ, assuming that the artery wall is isotropic and experiences isovolumetric change with pulse pressure.

Because of the constant ratio of wall thickness, h, to vessel radius, r, and blood density, ρ, PWV can be used as a direct correlation to arterial stiffness. With monitoring over time, changes in an individual's PWV can be directly linked to changes in arterial stiffness.

Alternately phrased, the Moens-Korteweg equation can state as follows:

PWV=√[(tE)/(ρd)],

where t is vessel wall thickness, ρ is blood density, d is the interior diameter of the vessel. As previously stated, PWV also equals the length of the vessel (L) traveled by the pulse divided by the PTT (T):

PWV=L/T.

The elastic modulus, E, is indicated as:

E=E₀e^{αP},

wherein E₀ is the modulus at zero pressure, α is dependent on the vessel, and P is the blood pressure. Making the appropriate combinations and substitutions into the Moens-Korteweg equation yields:

L/T=√[(tE₀e^{αP})/(ρd)],

which leads to:

P=(1/α[ln(L²ρd/tE₀)−(2 ln T)].

If changes to wall thickness t and diameter of the vessel d with respect to changes to blood pressure P are negligible, and the change in the modulus E₀ is slow enough, the change in blood pressure can be linearly related to the change in PTT as follows:

ΔP=(−2/αT)ΔT.

Similarly, through the Bramwell-Hill equation:

PWV=√[(ΔP·V)/(ρ·ΔV)],

where V is blood volume, ΔV is change in blood volume, ΔP is change in blood pressure, and ρ is blood density.

As found in the prior art through both the Moens-Korteweg and Bramwell-Hill equations, both PWV and PTT have been established to have approximate linear relationships to systolic and diastolic or mean blood pressure (P), according to the following equations:

P=(1/α)(PWV−b), and

P=(1/n)(m−PTT),

where a, b, m, and n are user or patient-specific constants.

The preferred embodiments given are meant as examples of the invention only, and not to unduly limit its scope. Those of ordinary skill in the arts of cardiovascular health and mechanical engineering will recognize that numerous variations are possible without escaping the inventive scope of the present invention. The scope of the invention is captured by the following claims:

1. A system for monitoring cardiovascular health comprising:
a) an electrode-based module, comprising at least two electrodes which contact skin, to generate a first set of data measuring a target heart beat;
b) a mobile device, held in a hand, comprising:
i) an optical module, comprising a camera, to generate a second set of data measuring a pulse wave created by the same target heart beat corresponding to the first set of data;
ii) a processor, in communication with both the electrode-based module and the optical module, that receives the first and second sets of data and calculates at least one cardiovascular health value using the data; and
iii) a wireless transceiver, operably connected to the processor, for data exchange.

2. The system of claim 1, wherein the cardiovascular health value is at least one of arterial stiffness, blood pressure, heart rate, pulse transit time, and pulse wave velocity.

3. The system of claim 1, wherein the mobile device is one of a smartphone, cell phone, tablet, personal digital assistant (PDA), handheld console, digital camera, and digital camcorder.

4. The system of claim 1, wherein the wireless transceiver utilizes Bluetooth technology.

5. The system of claim 1, wherein the electrode-based module is a chest-worn electrocardiographic source with a wireless transmitter.

6. The system of claim 1, wherein the electrode-based module is a plug-in ECG adaptor for the mobile device, said adaptor comprising at least two surface, contact electrodes and a means to plug into the mobile device.

7. The system of claim 1, wherein the electrode-based module comprises at least two surface, contact electrodes integrated with the mobile device.

8. The system of claim 1, wherein there are two electrode-based modules, one of which is a chest-worn electrocardiographic source with a wireless transmitter, and the other of which comprises surface, contact electrodes connected to the mobile device.

9. The system of claim 1, wherein the optical module is a photoplethysmographic source.

10. The system of claim 1, wherein the mobile device further comprises an accelerometer in communication with the processor.

11. An ECG adaptor, for a mobile device, comprising:
a. a housing;
b. at least two contact electrodes on the surface of the housing;
c. a processor within the housing;
d. an analog-to-digital converter within the housing which converts the analog signal from the contact electrodes into a digital signal that is conveyed to the processor; and
e. a means to plug into the mobile device and allow the adaptor's processor to communicate with the mobile device's processor.

12. The adaptor of claim 11, wherein the mobile device is one of a smartphone, cell phone, tablet, personal digital assistant (PDA), handheld console, digital camera, and digital camcorder.

13. The adaptor of claim 11, wherein the adaptor is plugged into the main bus port of the mobile device.

14. A method of monitoring at least one cardiovascular health marker over time comprising the following steps, in interchangeable order where possible:
a. obtaining an ECG signal for a target heart beat to generate a first set of data;
b. obtaining a PPG signal for a pulse wave from the same target heart beat, using a mobile device having a camera which functions as a PPG source, to generate a second set of data;
c. transmitting the first and second sets of data to the mobile device's processor;
d. calculating at least one cardiovascular health marker with the processor, using the first and second sets of data;
e. monitoring at least one cardiovascular health marker continuously over time.

15. The method of claim 14, wherein the cardiovascular health marker is at least one of arterial stiffness, blood pressure, heart rate, pulse transit time, and pulse wave velocity.

16. The method of claim 14, wherein the ECG signal is obtained via a wireless ECG source strapped around a user's chest.

17. The method of claim 14, wherein the ECG signal is obtained via ECG contact electrodes on the mobile device.

18. The method of claim 14, wherein the ECG signal is obtained via ECG contact electrodes on an adaptor connected to the mobile device.

19. The method of claim 14, further comprising a step wherein a second ECG signal for a target heart beat is obtained to generate a third set of data that is also transmitted to the processor and may be used to calibrate the first and second sets of data, with a synchronized system of two ECG and one PPG sources.

20. The method of claim 14, further comprising a step wherein streams of prolonged uninterrupted data measurements of at least 24 hours for each data stream, are collected across independent sequential date ranges and compared with each other to determine rate of change in at least one cardiovascular health marker.
